(19) United States
(12) Patent Application Publication
(10) Pub. No.: US 2017/0224549 A1
(43) Pub. Date: Aug. 10, 2017

(54) FLUID ABSORBENT CLOTHING

(71) Applicant: Melvin Frainklin Helit, Oceanside, CA (US)

(72) Inventor: Melvin Frainklin Helit, Oceanside, CA (US)

(21) Appl. No.: 15/268,293

(22) Filed: Sep. 16, 2016

Related U.S. Application Data
(60) Provisional application No. 62/292,683, filed on Feb. 8, 2016.

(51) Int. Cl. A61F 13/49; A61F 13/56; A61F 13/505; A41B 9/00; A61F 13/496

(52) U.S. Cl. CPC A61F 13/49061; A41B 9/001; A41B 9/004; A61F 13/496; A61F 13/505; A61F 13/5655; A61F 2013/15121

(57) ABSTRACT

A fluid absorbent clothing is configured to absorb fluid from a human user. The fluid absorbent clothing includes a waistband, attached to a front panel and a rear panel. An interior pocket panel is attached to an inside of the front panel forming an interior pocket. A removable desiccant is inserted into the interior pocket panel. Fluid excreted from the human user is absorbed by the removable desiccant making the human user dry.

### RELATED APPLICATION

This application claims priority to provisional patent application U.S. Ser. No. 62/292,683 filed on Feb. 8, 2016, the entire contents of which is herein incorporated by reference.

### BACKGROUND

The embodiments herein relate generally to clothing worn by a human user.

Prior to embodiments of the disclosed invention, diapers provided fluid absorbency but were uncomfortable whereas briefs provided comfort but lacked fluid absorbency. Embodiments of the disclosed invention solve these problems.

### SUMMARY

A fluid absorbent clothing is configured to absorb fluid from a human user. The fluid absorbent clothing includes a waistband, attached to a front panel and a rear panel. An interior pocket panel is attached to an inside of the front panel forming an interior pocket. A removable desiccant is inserted into the interior pocket panel. Fluid excreted from the human user is absorbed by the removable desiccant making the human user dry.

### BRIEF DESCRIPTION OF THE FIGURES

The detailed description of some embodiments of the invention is made below with reference to the accompanying figures, wherein like numerals represent corresponding parts of the figures.

FIG. 1 shows a front perspective view of one embodiment of the present invention;

FIG. 2 shows a rear perspective view of one embodiment of the present invention;

FIG. 3 shows a front perspective view of one embodiment of the present invention in inside-out configuration;

FIG. 4 shows a rear perspective view of one embodiment of the present invention in inside-out configuration;

FIG. 5 shows an assembly view of one embodiment of the present invention;

FIG. 6 shows a section view of one embodiment of the present invention;

FIG. 7 shows a section view of one embodiment of the present invention taken along line 7-7 in FIG. 6; and

FIG. 8 shows a section view of one embodiment of the present invention.

### DETAILED DESCRIPTION OF CERTAIN EMBODIMENTS

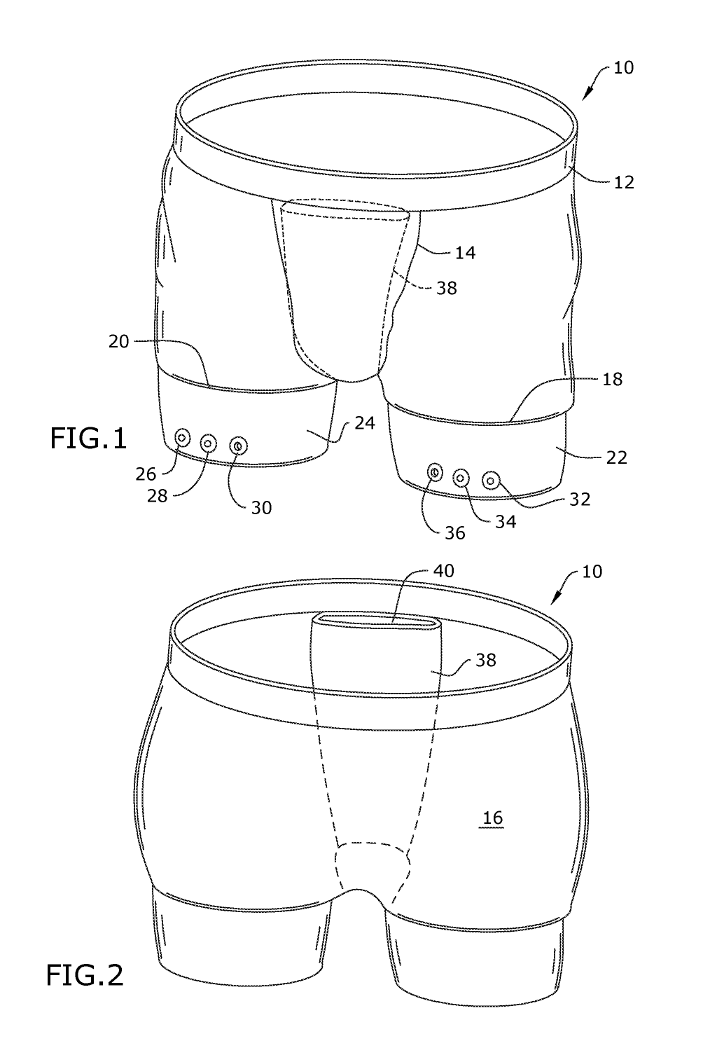

By way of example, and referring to FIG. 1 and FIG. 2, one embodiment of fluid absorbent clothing 10 comprises waistband 12 attached to front panel 14 and rear panel 16. Rear panel 16 is formed into first leg opening 18 and second leg opening 20. First leg opening 18 is sewn to first leg extension 22. Likewise, second leg opening 20 is sewn to second leg extension 24.

First leg extension 22 is attached to first leg extension first male fastener 26, first leg extension second male fastener 28, and first leg extension first female fastener 30. Second leg extension 24 is attached to second leg extension first male fastener 32, second leg extension second male fastener 34, and second leg extension first female fastener 36.

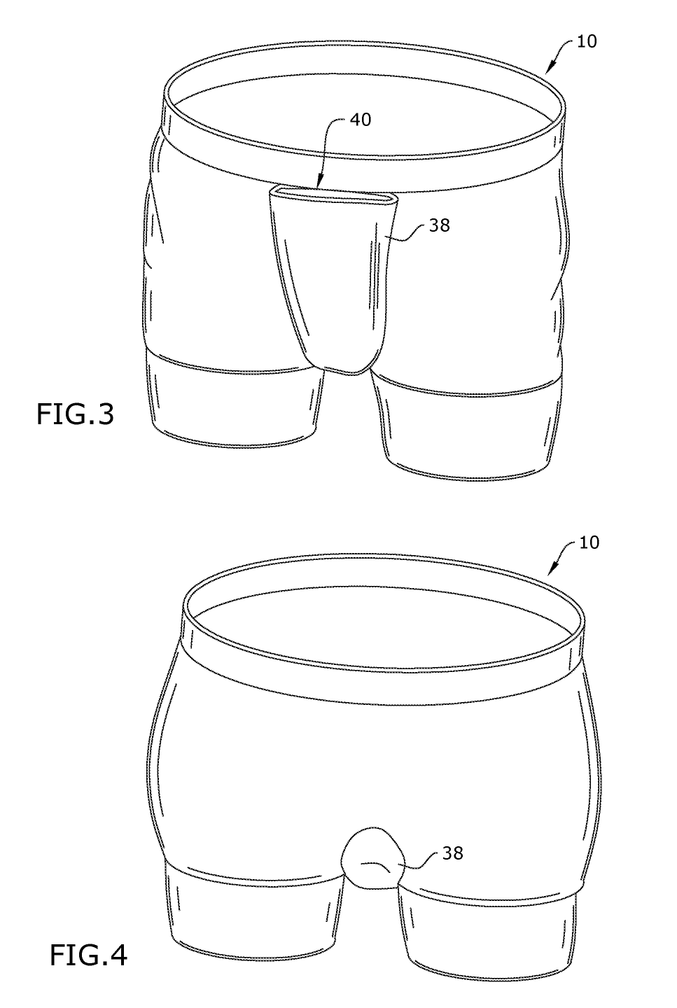

Turning to FIG. 3 and FIG. 4, an inside of front panel 14 is attached to interior pocket panel 38. This forms interior pocket 40 between front panel 14 and interior pocket panel 38.

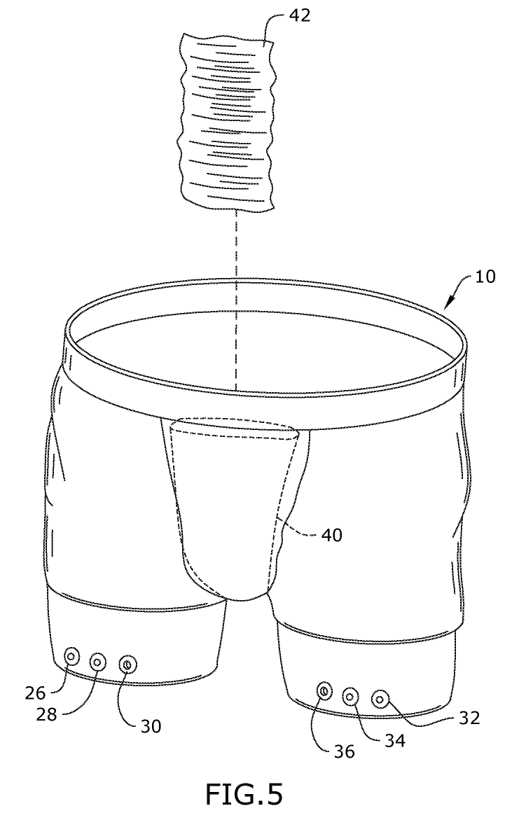

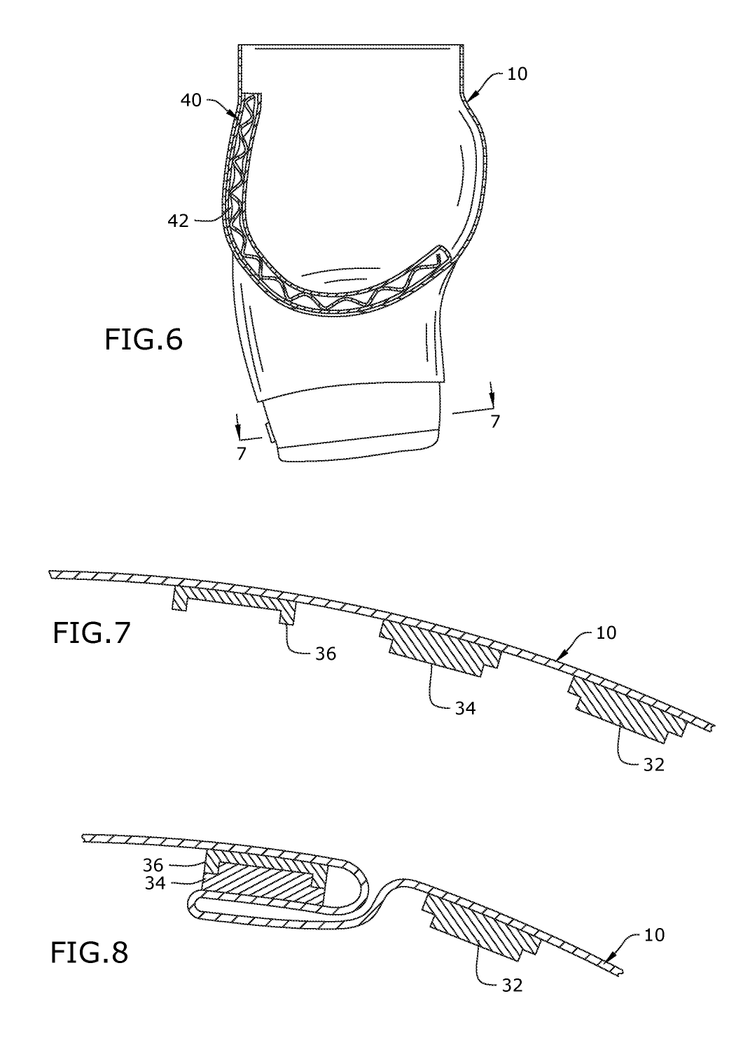

As shown in FIG. 5 and FIG. 6 in a first mode of operation, removable desiccant 42 is inserted into interior pocket 40. This absorbs fluid and can be removed by the user. At this point, a second removable desiccant 42 can be inserted into interior pocket 40 and so forth. Tissue paper can be used as a removable desiccant as well as many other materials.

Turning to FIG. 7 and FIG. 8, in a second mode of operation a user can join first leg extension first male fastener 26 to first leg extension first female fastener 30 to secure overflow waste in first leg extension 22. Similarly, a user can join second leg extension first male fastener 32 to second leg extension first female fastener 36 to secure overflow waste in second leg extension 24.

In some embodiments, waistband 12 can be made from a 1.5-inch-wide Lycra flat straight cut stretch binding, a variety of which is sold under the trademark SPANDEX. Interior pocket panel 38 has an upper edge that can be seven inches in length, and a side length that can be 12 inches long. Interior pocket panel 38 should be made of nylon.

In some embodiments, interior pocket panel 38 covers a radial portion of fluid absorbent clothing 10 that is at least 90 degrees but no more than 180 degrees. In some embodiments, this radial portion can be about 135 degrees.

As used in this application, the term “a” or “an” means “at least one” or “one or more.”

As used in this application, the term “about” or “approximately” refers to a range of values within plus or minus 10% of the specified number.

As used in this application, the term “substantially” means that the actual value is within about 10% of the actual desired value, particularly within about 5% of the actual desired value and especially within about 1% of the actual desired value of any variable, element or limit set forth herein.

All references throughout this application, for example patent documents including issued or granted patents or equivalents, patent application publications, and non-patent literature documents or other source material, are hereby incorporated by reference herein in their entireties, as though individually incorporated by reference, to the extent each reference is at least partially not inconsistent with the disclosure in the present application (for example, a reference that is partially inconsistent is incorporated by reference except for the partially inconsistent portion of the reference).

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

Any element in a claim that does not explicitly state “means for” performing a specified function, or “step for” performing a specified function, is not to be interpreted as a “means” or “step” clause as specified in 35 U.S.C. §112, ¶ 6. In particular, any use of “step of” in the claims is not intended to invoke the provision of 35 U.S.C. §112, ¶ 6.

Persons of ordinary skill in the art may appreciate that numerous design configurations may be possible to enjoy the functional benefits of the inventive systems. Thus, given the wide variety of configurations and arrangements of embodiments of the present invention the scope of the invention is reflected by the breadth of the claims below rather than narrowed by the embodiments described above.

What is claimed is:

1. A fluid absorbent clothing, configured to absorb fluid from a human user; the fluid absorbent clothing comprising:
a waistband, attached to a front panel and a rear panel;
an interior pocket panel, attached to an inside of the front panel forming an interior pocket;
a removable desiccant, inserted into the interior pocket panel;
wherein fluid excreted from the human user is absorbed by the removable desiccant making the human user dry.

2. The fluid absorbent clothing of claim 1, further comprising: a first leg opening and a second leg opening, formed from the rear panel.

3. The fluid absorbent clothing of claim 2, further comprising:
a first leg extension, sewn to the first leg opening; and
a second leg extension, sewn to the second leg opening.

4. The fluid absorbent clothing of claim 3, further comprising:
a first leg extension first male fastener, a first leg extension second male fastener, and a first leg extension first female fastener, attached to the first leg extension; wherein joining the first leg extension first male fastener to the first leg extension first female fastener secures overflow waste in the first leg extension; and
a second leg extension first male fastener, a second leg extension second male fastener, and a second leg extension first female fastener, attached to the second leg extension; wherein joining the second leg extension first male fastener to the second leg extension first female fastener secures overflow waste in the second leg extension.
